(19) United States
(12) Patent Application Publication
(10) Pub. No.: US 2024/0226066 A1
(43) Pub. Date: Jul. 11, 2024

(54) CARDIOPROTECTION BY AUTOPHAGY INDUCTION AND METABOLIC REPROGRAMMING

(71) Applicants: UNIVERSITE PARIS-SACLAY, GIF-SUR-YVETTE (FR); INSERM (INSTITUT NATIONAL DE LA SANTÉ ET DE LA RECHERCHE MÉDICALE), PARIS (FR); COMMISSARIAT A L'ENERGIE ATOMIQUE ET AUX ENERGIES ALTERNATIVES, PARIS (FR); INSTITUT GUSTAVE ROUSSY, VILLEJUIF (FR)

(72) Inventors: CATHERINE BRENNER, LE CHESNAY (FR); FRÉDÉRIC TARAN, GIF-SUR-YVETTE (FR); JEAN-CHRISTOPHE CINTRAT, IGNY (FR); JEAN-LUC PERFETTINI, MEAUX (FR)

(21) Appl. No.: 18/288,592

(22) PCT Filed: Apr. 29, 2022
(86) PCT No.: PCT/FR2022/050829

§ 371 Date: Oct. 27, 2023

(30) Foreign Application Priority Data
Apr. 30, 2021 (FR) FR2104564

(51) Int. Cl. A61K 31/4174; A61K 31/15; A61K 31/513; A61P 9/14

(52) U.S. Cl. CPC A61K 31/4174; A61K 31/15; A61K 31/513; A61P 9/14

(57) ABSTRACT

The invention relates to compounds capable of inducing autophagy and metabolic reprogramming and to the use thereof as cardioprotectors, in particular in the context of anticancer therapy.

### TECHNICAL FIELD

The invention relates to compounds capable of inducing autophagy and metabolic reprogramming and to the use thereof as cardioprotectors, in particular in the context of anticancer therapy.

### TECHNOLOGICAL BACKGROUND

Despite recent advances in anticancer therapeutic strategies (e.g. radiotherapy, chemotherapy and immunotherapy), these strategies are associated with a high risk of cardiac complications, including heart failure or cardiomyopathies in many surviving patients, resulting in increased cardiovascular morbidity and mortality. Moreover, the general ageing of the population is accompanied by an increase in the number of heart diseases involving an excess of cell death, in particular myocardial infarction, ischemia-reperfusion and heart failure. In order to prevent cardiotoxicity, a strategy combining anticancer therapy with molecules which possess cardioprotective activity has been developed. For example, the FDA (Food and Drug Administration) has authorized the administration of dexrazoxane, an antioxidant, to patients to counteract the effects of anticancer strategies. However, it has since been observed that the use of dexrazoxane in this context is associated with a higher rate of secondary malignant neoplasms (Shaikh et al., J Natl Cancer Inst., 2016, 108(4)). There is therefore still an urgent need for molecules capable of preventing long-term cardiotoxicity.

### SUMMARY OF THE INVENTION

The inventors have developed a high-throughput screening test to identify inhibitors of the main cardiac cell death modalities, that is to say H₂O₂-induced necrosis and camptothecin-induced apoptosis, among chemical libraries composed of 1600 molecules, on a H9C2 cardiomyoblast line. This test has thus made it possible to identify cardioprotective molecules capable of compensating for the cardiotoxic effects of anticancer therapeutic strategies. The inventors have been able to show that the molecules identified in the context of the present invention act by inhibiting the death of cardiomyocytes by induction of autophagy and metabolic reprogramming resulting in inhibition of the apoptosis and necrosis phenomena. The molecules identified can in particular be used in combination with chemotherapy or radiotherapy to protect cardiac cells and reduce the side effects of anticancer treatments.

Thus, according to a first aspect, the invention relates to such cardioprotective molecules capable of inducing autophagy and metabolic reprogramming, for the use thereof in the prevention of cardiac side effects of an anticancer treatment.

More particularly, the invention relates to a compound selected from the group consisting of SG6163F, LOPA87 and VP331, and analogs thereof, for the use thereof as a cardioprotector. According to an embodiment, the compound is selected from SG6163F and LOPA87, and analogs thereof, more particularly from SG6163F and LOPA87.

According to one embodiment, the compound is used in the treatment of a heart disease selected from myocardial infarction, ischemia-reperfusion and heart failure.

According to another embodiment, the compound is used in cardioprotection against the cardiotoxic side effects of a therapy inducing cardiotoxic side effects, in particular anticancer therapy. In particular, the invention relates to a compound selected from the group consisting of SG6163F, LOPA87 and VP331, and analogs thereof, in combination with a therapeutic agent such as an anticancer agent, for use thereof in cardioprotection against the cardiotoxic side effects of a therapy, such as cancer therapy, which induces cardiotoxic side effects.

According to a particular embodiment, the compound can be administered before, during or after the therapy inducing cardiotoxic side effects.

According to another aspect, the invention relates to a kit of components comprising:
(a) a compound selected from the group consisting of SG6163F, LOPA87 and VP331, and analogs thereof; and
(b) a therapeutic agent useful in treating a patient, said agent however inducing cardiotoxic side effects;
   wherein component (a) is intended to be used for its cardioprotective effect against cardiotoxic side effects of component (b).

According to a particular embodiment, components (a) and (b) are suitable for sequential, separate and/or simultaneous administration.

According to another aspect, the kit according to the invention is used in the context of the treatment of cancer, component (b) being a chemotherapeutic or immunotherapeutic anticancer agent, and component (a) being used for its cardioprotective effect against the cardiotoxic side effects of component (b).

According to another aspect, the invention relates to a method for screening for compounds which have cardioprotective activity, or which may have cardioprotective activity, said method comprising the following steps:
(a) culturing cardiac cells in a culture medium containing a test compound;
(b) culturing the cardiac cells in a culture medium containing a cell death inducer; and
(c) measuring cell viability.

### DETAILED DESCRIPTION

According to the invention, the compounds used are compounds capable of inducing autophagy and metabolic reprogramming and of inhibiting cardiomyocyte death by inhibiting apoptosis and necrosis phenomena. The inventors have been able to demonstrate that such compounds are cardioprotective, without the effect of inducing cancer cell proliferation.

### Compound Capable of Inducing Autophagy and Metabolic Reprogramming and Use as a Cardioprotector

The invention therefore relates to a compound capable of inducing autophagy and metabolic reprogramming, for use thereof as a cardioprotector. More particularly, the invention relates to a compound capable of inducing autophagy and metabolic reprogramming and of inhibiting cardiomocyte death by inhibiting apoptosis and/or necrosis, for use thereof as a cardioprotector.

Autophagy plays an essential role in cellular homeostasis. It is a catabolic process conserved in evolution that involves the degradation of damaged cytoplasmic components. The inventors have been able to demonstrate that this autophagy mechanism can advantageously be used to prevent cardiotoxic effects, in particular cardiotoxic effects induced by anticancer treatments.

Those skilled in the art are well aware of the autophagy mechanism and the means enabling them to identify compounds capable of inducing this mechanism. Moreover, they will be able to use the process presented in the experimental section of the present application to identify other compounds possessing such properties.

According to a particular embodiment, the compound capable of inducing autophagy is a compound selected from digitoxigenin, digoxin, minaprine, SG6163F, VP331 and LOPA87, and analogs thereof. The ability of these compounds to induce autophagy in cardiac cells and metabolic reprogramming by modulation of glycolysis and mitochondrial respiration involving an effect on the mitochondrial network had never been reported in the prior art. These properties newly identified in the context of the present invention can advantageously be used in order to induce a cardioprotective effect in patients requiring such cardioprotection.

The formulae of the digitoxigenin, digoxin, minaprine, SG6163F, VP331 and LOPA87 compounds are recalled below:

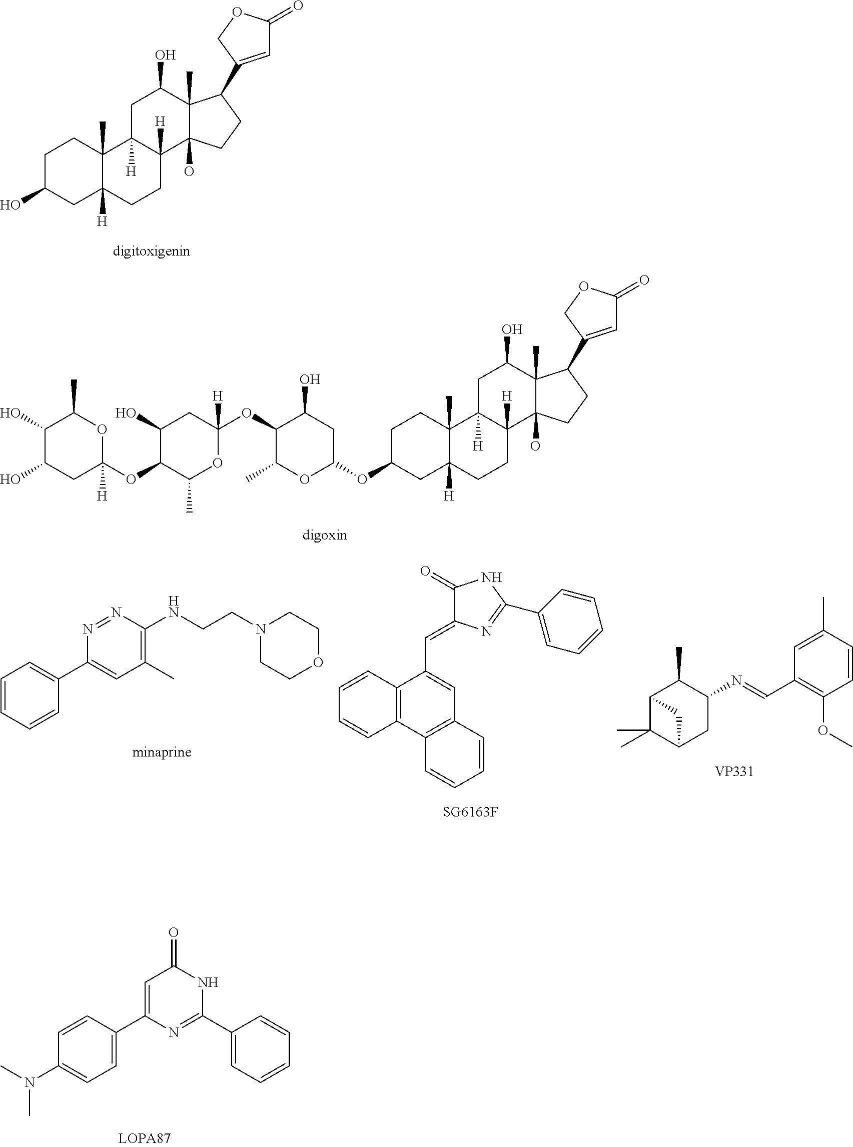

Preferably, the compound is selected from compounds SG6163F, VP331 and LOPA87, and analogs thereof. More preferably, the compound is selected from compounds SG6163F and LOPA87, and analogs thereof.

According to a particular embodiment, the analog of the SG6163F compound is a compound of formula (I):

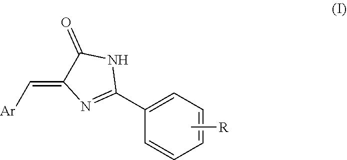

wherein:

Ar is a C₆-C₁₄ aryl group or a C₅-C₁₀ heteroaryl group, said aryl or heteroaryl group being optionally substituted with 1 to 5 groups selected from C₆-C₁₄ aryl groups, C₅-C₁₀ heteroaryl groups, halogen atoms, C₁-C₆ alkyl groups, a hydroxyl group, C₁-C₆ alkoxyl groups, an NH₂ group, an NO₂ group, mono(C₁-C₆)alkylamino groups and di(C₁-C₆)alkylamino groups; and

R is selected from C₆-C₁₄ aryl groups, C₅-C₁₀ heteroaryl groups, halogen atoms, C₁-C₆ alkyl groups, a hydroxyl group, C₁-C₆ alkoxyl groups, an NH₂ group, an NO₂ group, mono(C₁-C₆)alkylamino groups and di(C₁-C₆)alkylamino groups.

According to a particular embodiment, the Ar group is a C₆-C₁₀ aryl group. According to another particular embodiment, the C₆-C₁₀ aryl group is selected from phenyl, fluorenyl, anthracenyl and naphthyl groups. According to another particular embodiment, Ar is an unsubstituted aryl group, in particular an unsubstituted phenyl or naphthyl group, more particularly an unsubstituted phenyl group. According to a variant embodiment, Ar is a C₆-C₁₀ aryl group substituted with 1 to 5 groups as defined above. According to another embodiment, Ar is a C₆-C₁₀ aryl group substituted with a group selecred from C₆-C₁₄ aryl groups, C₅-C₁₀ heteroaryl groups, halogen atoms, C₁-C₆ alkyl groups, a hydroxyl group, C₁-C₆ alkoxyl groups, an NH₂ group, an NO₂ group, mono(C₁-C₆)alkylamino groups and di(C₁-C₆)alkylamino groups. According to another embodiment, Ar is a C₆-C₁₀ aryl group substituted by a group selected from C₆-C₁₄ aryl groups, in particular phenyl, halogen atoms, C₁-C₆ alkyl groups and C₁-C₆ alkoxyl groups. According to another embodiment, Ar is a C₆-C₁₀ aryl group substituted by a group chosen from C₆-C₁₄ aryl groups, in particular phenyl, halogen atoms and C₁-C₆ alkoxyl groups. According to another embodiment, Ar is a phenyl group monosubstituted with a phenyl group, a halogen atom, in particular a fluorine atom, or a methoxyl group. According to one variant, Ar is a naphthyl group monosubstituted with a halogen atom or a methoxyl group. According to one variant, Ar is a naphthyl group monosubstituted with a methoxyl group.

According to a particular embodiment, the Ar group is a C₅-C₁₀ heteroaryl group. According to another particular embodiment, Ar is an unsubstituted heteroaryl group. According to a variant embodiment, Ar is a heteroaryl group substituted with 1 to 5 groups as defined above. According to another embodiment, Ar is a heteroaryl group substituted with a group selected from C₆-C₁₄ aryl groups, C₅-C₁₀ heteroaryl groups, halogen atoms, C₁-C₆ alkyl groups, a hydroxyl group, C₁-C₆ alkoxyl groups, an NH₂ group, an NO₂ group, mono(C₁-C₆)alkylamino groups and di(C₁-C₆)alkylamino groups. According to another embodiment, Ar is a heteroaryl group, in particular an imidazolyl group, substituted with a group selected from halogen atoms, C₁-C₆ alkyl groups and C₁-C₆ alkoxyl groups. According to another particular embodiment, Ar is a heteroaryl group, in particular an imidazolyl group, substituted with a C₁-C₆ alkyl group. According to one variant, Ar is a heteroaryl group, in particular an imidazolyl group, substituted with a methyl group.

According to another embodiment, R is selected from halogen atoms, C₁-C₆ alkyl groups, C₁-C₆ alkoxyl groups and the NO₂ group. According to a particular embodiment, R is selected from halogen atoms, C₁-C₆ alkoxyl groups and the NO₂ group. More particularly, R is selected from halogen atoms, more particularly the chlorine atom, the methoxyl group and the NO₂ group.

Among the specific analogs of the SG6163F compound which can be used in the context of the present invention, mention may be made of the SG6144, SG6146, SG6149, LOPA90, LOPA93, LOPA99, LOPA101, LOPA104, LOPA105 and LOPA106 compounds:

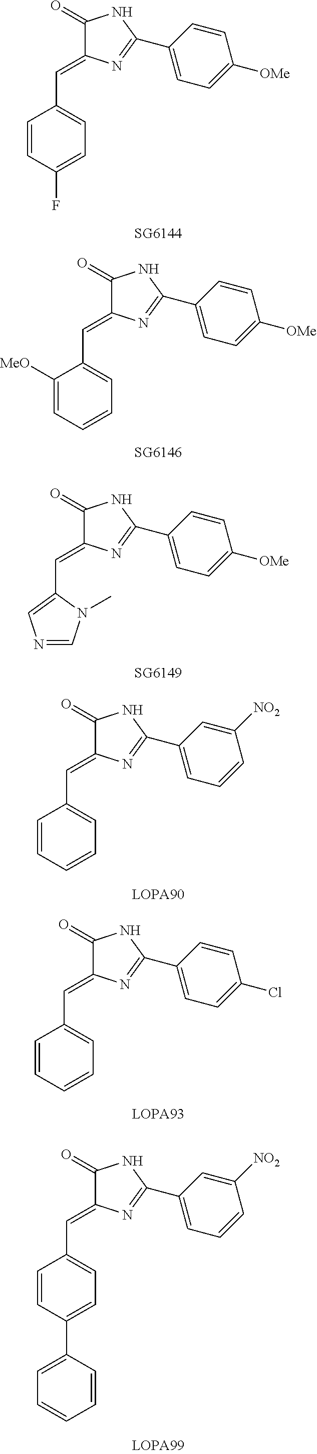 [SMILES: COc1ccc(C2=N/C(=C\c3ccc(F)cc3)C(=O)N2)cc1 ; COc1ccc(C2=N/C(=C\c3ccccc3OC)C(=O)N2)cc1 ; COc1ccc(C2=N/C(=C\c3cncn3C)C(=O)N2)cc1 ; O=C1NC(c2cccc([N+](=O)[O-])c2)=N/C1=C\c1ccccc1 ; O=C1NC(c2ccc(Cl)cc2)=N/C1=C\c1ccccc1 ; O=C1NC(c2cccc([N+](=O)[O-])c2)=N/C1=C\c1ccc(-c2ccccc2)cc1]

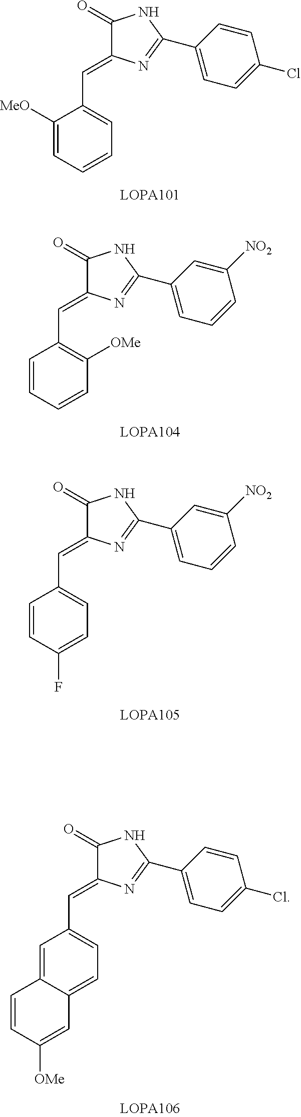 [SMILES: COc1ccccc1/C=C1\N=C(c2ccc(Cl)cc2)NC1=O ; COc1ccccc1/C=C1\N=C(c2cccc([N+](=O)[O-])c2)NC1=O ; O=C1NC(c2cccc([N+](=O)[O-])c2)=N/C1=C\c1ccc(F)cc1 ; COc1ccc2cc(/C=C3\N=C(c4ccc(Cl)cc4)NC3=O)ccc2c1]

According to a particular embodiment, the analog of the LOPA87 compound is a compound of formula (II):

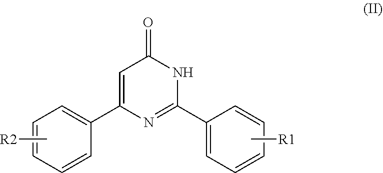

wherein:

R1 and R2, which may be identical or different, are selected from the following groups: hydrogen atom, halogen atom, C₆-C₁₄ aryl, C₁-C₆ alkyl, hydroxyl, C₁-C₆ alkoxyl, NH₂, NO₂, mono(C₁-C₆)alkylamino and di(C₁-C₆)alkylamino.

According to a particular embodiment, in the compound of formula (II), R1 is selected from hydrogen and a halogen atom. According to a particular embodiment, in the compound of formula (II), R1 is a hydrogen atom. According to another embodiment, in the compound of formula (II), R2 is a C₆-C₁₄ aryl, more particularly a phenyl group.

According to a particular embodiment, the analog of the LOPA87 compound is the LOPA86 compound:

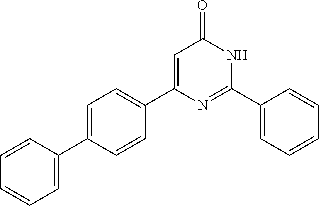 [SMILES: O=c1cc(-c2ccc(-c3ccccc3)cc2)nc(-c2ccccc2)[nH]1]

Those skilled in the art may refer to the article by Gabillet et al. for the synthesis of SG6163F and LOPA87 compounds and analogs thereof (Gabillet et al., J. Org. Chem. 2014, 79, pp. 9894-9898).

In a particular embodiment, the compound is selected from the VP331 compound and analogs thereof.

According to a particular embodiment, the analog of the VP331 compound is a compound of formula (III):

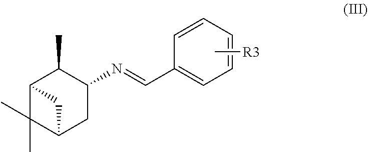

wherein R is selected from a C₆-C₁₄, in particular C₆-C₁₀, aryl group, more particularly a phenyl group; a halogen atom; a C₁-C₆ alkyl; OH; a C₁-C₆ alkoxyl; NH₂; NO₂; mono(C₁-C₆)alkylamino and di(C₁-C₆)alkylamino.

Those skilled in the art may refer to patent applications EP3476389 and EP3095688 for the synthesis of the VP331 compound and analogs thereof.

In another particular embodiment, the compound used is minaprine or a pharmaceutically acceptable salt thereof, more particularly minaprine dihydrochloride.

The compounds according to the invention can be used in the form of pharmaceutically acceptable salts, in particular salts of inorganic and organic acids. Representative examples of inorganic acids include hydrochloric acid, hydrobromic acid, hydroiodic acid, phosphoric acid, etc. Representative examples of organic acids include formic acid, acetic acid, trichloroacetic acid, trifluoroacetic acid, propionic acid, benzoic acid, cinnamic acid, citric acid, fumaric acid, maleic acid, methanesulfonic acid, etc. Other organic or inorganic acid addition salts include the pharmaceutically acceptable salts described in J. Pharm. Sci. 1977, 66, 2, and in the “Handbook of Pharmaceutical salts: Properties, Selection, and Use” edited by P. Heinrich Stahl and Camille G. Wermuth 2002.

The compounds used according to the present invention may be incorporated into a pharmaceutical composition comprising, in a pharmaceutically acceptable carrier, at least one compound according to the invention as described above, optionally in combination with another therapeutic active agent.

The expression “pharmaceutically acceptable carrier” is understood to mean any carrier (for example excipient, substance, solvent, etc.) which does not interfere with the efficacy of the biological activity of the therapeutic active agent(s) present in the pharmaceutical composition and which is not toxic to the patient to whom the composition is administered. The pharmaceutical compositions according to the invention advantageously comprise one or more pharmaceutically acceptable excipients or vehicles. Mention may be made, for example, of saline, physiological, isotonic, buffered solutions, etc. compatible with pharmaceutical use and known to those skilled in the art. The compositions may contain one or more agents or vehicles selected from dispersants, solubilizers, stabilizers, preservatives, solvents, etc.

The compounds or compositions according to the invention can be administered in different ways and in different forms. Thus, they can be administered orally or systemically, such as for example intravenously, intramuscularly, subcutaneously, transdermally, intra-arterially, intracerebrally, etc. For injections, the compounds are generally packaged in the form of liquid suspensions, which can be injected by means of syringes or infusions, for example. It is understood that the injected flow rate and/or dose can be adjusted by those skilled in the art according to the patient, the pathology, the mode of administration, etc. Typically, the compounds are administered at doses which can range between 1 μg and 2 g/administration, preferentially from 0.1 mg to 1 g/administration. The administrations may be daily or repeated several times a day, as appropriate.

Moreover, the composition according to the invention may also comprise at least one other therapeutic active ingredient or agent.

The compounds according to the invention, by virtue of their cardioprotective activity, can be advantageously used in the treatment of a heart disease. In particular, the heart disease may be myocardial infarction, ischemia-reperfusion or heart failure.

The term “treatment” or “treat” means, according to the invention, an improvement in or prophylaxis of the disorder or of the disease, or of at least one of its symptoms. This includes the improvement or prevention of at least one measurable physical parameter of the disease to be treated, which is not necessarily discernible in the subject. The term “treatment” or “treat” also refers to the inhibition or slowing of the progression of the disease, or the stabilization of one of the symptoms of the disease. It may also be a delay in the triggering at least one symptom of the disease. According to some embodiments, the compounds of the invention are administered as a preventive measure. In this context, the term “treatment” or “treat” refers to the reduction of the risk of developing the disease.

The compounds according to the invention are particularly suitable for preventing the cardiotoxic effects of certain therapeutic strategies. The invention therefore also relates to a compound according to the invention, for use thereof as a cardioprotector against the cardiotoxic side effects of a therapy administered to the patient, said therapy being capable of inducing said cardiotoxic effects. The invention also relates to a compound according to the invention, for use thereof in a method of preventing the cardiotoxic effects of a treatment administered to a patient. Thus, advantageously, the compounds of the invention can in particular be used in a combined treatment, in order to advantageously capitalize on their cardioprotective properties. A combined treatment comprises the administration of a compound according to the invention in combination with a therapeutic strategy aimed at the treatment of a pathology from which the patient is suffering, said therapeutic strategy being capable of having cardiotoxic effects. According to one embodiment, the compound of the invention is administered to a patient suffering from cancer, to whom an anticancer treatment is administered.

According to the present invention, the expression “anticancer treatment”, or its variants, is intended for a treatment used to slow the growth of or destroy cancer cells. The anticancer treatment may be drug-based or non-drug-based. Among the drug-based anticancer treatments, mention may be made in particular of chemotherapies and immunotherapies.

The term “chemotherapy” refers to a type of cancer treatment that uses one or more anticancer drugs (“chemotherapeutic agents”). The chemotherapy may be administered with a curative aim or may be intended to prolong life or reduce the symptoms of a cancer from which the patient is suffering. The chemotherapeutic agents are, for example, selected from anticancer alkylating agents, anticancer antimetabolites, anticancer antibiotics, plant-derived anticancer agents, anticancer platinum coordination compounds, and any combination thereof. Among the chemotherapeutic agents which can be used in the context of the invention, the cardiotoxic effect of which can advantageously be prevented by means of the compounds of the invention, mention may be made of anthracyclines, in particular doxorubicin, daunorubicin, epirubicin, idarubicin, more particularly doxorubicin, tyrosine kinase inhibitors such as imatinib, taxanes (e.g. docetaxel, paclitaxel), anthracenediones (e.g. mitoxantrone (MTX), PARP inhibitors, antimetabolites such as methotrexate and anti-HER2 agents such as trastuzumab.

The term “immunotherapy” refers to a therapy aimed at inducing and/or enhancing an immune response to a specific target, for example to cancer cells. Immunotherapy may involve the use of checkpoint inhibitors, checkpoint agonists (also known as T lymphocyte agonists), IDO inhibitors, PI3K inhibitors, adenosine receptor inhibitors, adenosine-producing enzyme inhibitors, adoptive transfer, therapeutic vaccines, and combinations thereof. Among the immunotherapeutic agents which can be used in the context of the invention, the cardiotoxic effect of which can advantageously be prevented by means of the compounds of the invention, mention may be made of anti-PD-1 antibodies and anti-CTLA-4 antibodies. According to a particular embodiment, the immunotherapeutic treatment is a combination of an anti-PD-1 antibody and an anti-CTLA-4 antibody.

Radiotherapy may be mentioned, in a non-limiting manner, among non-drug-based treatments. The expression “radiotherapy” refers to a locoregional treatment of cancers using radiation, in particular X-rays, gamma rays, neutron radiation, an electron beam, a proton beam and radiation sources, to destroy the cancer cells by blocking their ability to multiply. Any form of radiotherapy can be used in the context of the present invention, in particular external radiotherapy, internal radiotherapy (or “brachytherapy”), radioimmunotherapy, or metabolic radiotherapy involving oral administration or administration by intravascular (in particular intravenous) injection, of a radioactive substance that binds preferentially to the cancer cells to destroy them.

The compounds according to the invention are administered to any patient who will benefit or who is likely to benefit from their cardioprotective effect. The patient may have a cancer of any stage, in particular an early non-invasive cancer or a late-stage cancer that has already progressed to form metastases in the body.

The term “cancer” is understood to mean any form of cancer or tumors. Nonlimiting examples of cancers include brain cancer (for example glioma), gastric cancer, head and neck cancer, pancreatic cancer, non-small cell lung cancer, small cell lung cancer, prostate cancer, colon cancer, non-hodgkin's lymphoma, sarcoma, testicular cancer, acute non-lymphocytic leukemia and breast cancer. In a particular embodiment, the brain cancer is an astrocytoma, and more particularly glioblastoma multiforme; the lung cancer is either a small cell lung carcinoma or a small cell lung carcinoma; and the head and neck cancer is squamous cell carcinoma or adenocarcinoma. According to another embodiment, the cancer is a pediatric cancer. By way of illustration, among these pediatric cancers, mention may be made of neuroblastomas, Ewing's sarcoma, osteosarcomas, medulloblastomas, rabdomyosarcomas and pediatric glioblastomas.

The compounds may be administered before, at the same time as, or after the anticancer treatment. The compound or the pharmaceutical composition according to the invention is more specifically for simultaneous, separate or sequential use or administration of the compound according to the invention and of at least one other therapeutic active ingredient or agent or any other non-drug-based therapeutic strategy. Thus, the invention also relates to a kit of components comprising:
(a) a compound selected from the group consisting of digitoxigenin, digoxin, minaprine SG6163F, LOPA87 and VP331, and analogs thereof; and
(b) a chemotherapeutic or immunotherapeutic agent;
   wherein component (a) is used for its cardioprotective effect against the cardiotoxic side effects of component (b).

In a particular embodiment, component (a) is selected from SG6163F, LOPA87 and VP331, and analogs thereof, more particularly from SG6163F and LOPA87, and analogs thereof, preferentially from SG6163F and LOPA87. Preferably, component (a) is the SG6163F compound. The kit of components according to the invention comprises in particular components (a) and (b) suitable for sequential, separate and/or simultaneous administration. Advantageously, the kit of components according to the invention can be used in the treatment of a cancer, component (a) being used for its cardioprotective effect against the cardiotoxic side effects of component (b).

### Method for Screening for Cardioprotective Compounds

A second aspect of the invention relates to a method for screening for compounds which may have cardioprotective activity, said method comprising the following steps:
(a) culturing cardiac cells in a culture medium containing a test compound;
(b) culturing the cardiac cells in a culture medium containing a cell death inducer; and
(c) measuring cell viability.

The cells used are selected from cardiac cells known to those skilled in the art, in particular from primary cardiac cells or cardiac cell lines. According to a particular embodiment, the cardiac cells are rodent cells, in particular rat or mouse cells, more particularly rat cells. For example, the cells are H9C2 cells.

The cells are cultured in a medium suitable for their culture. Mention may in particular be made of DMEM medium supplemented with suitable additives. Among the additives which can be used, mention may be made of fetal calf serum, glutamine and antibiotics, in particular for the culture of H9C2 cells.

In step a), the culture medium is also supplemented with a test compound. According to a particular embodiment, the culture medium containing the test compound does not contain fetal calf serum and/or any antibiotic. In particular, according to a variant of the invention, the culture medium containing the test compound does not contain fetal calf serum and does not contain any antibiotic. The term “test compound” is understood to mean a compound for which the intention is to determine whether it possesses cardioprotective activity. The test compound will be used at a concentration capable of making it possible to identify said activity. Of course, those skilled in the art may choose to use a concentration range during the method according to the invention in order to identify the activity of the compound and the effective concentration. The cells are cultured in the presence of the test compound for a sufficient time to allow said test compound to act on the cells. By way of illustration, the culturing of the cells in the presence of the test compound can be carried out for a time of between 1 minute and 24 hours, for example between 15 minutes and 10 hours, in particular between 1 hour and 3 hours, more particularly for 2 hours.

In step b), the cells are cultured in a complete medium comprising serum and antibiotics, which also comprises a cell death inducer. According to a particular embodiment, in step b), the cell death inducer is introduced into the culture medium of step a). According to another embodiment, in step b), the culture medium of step a) is replaced with a culture medium containing a cell death inducer and the test compound. According to another, preferred, embodiment, in step b), the culture medium of step a) is replaced with a culture medium containing a cell death inducer but not containing the test compound. Among the cell death inducers, mention may be made of apoptosis inducers or necrosis inducers. Camptothecin may be mentioned among the apoptosis inducers which can be used in the context of the method according to the invention. Hydrogen peroxide (H₂O₂) can be used as a necrosis inducer. The cells are cultured in the presence of the cell death inducer at a concentration and for a time suitable for observing cell death in the cells not treated with a test compound, and for observing an absence or reduction of cell death in cells treated with a test compound which is found to have cardioprotective activity. For example, camptothecin can be used at a concentration of between 1 and 100 μM, in particular between 5 and 15 μM, more particularly 10 μM, for a period ranging from 10 h to 14 h, in particular 12 h to 36 h, more particularly 24 h. According to another example, the hydrogen peroxide is used for 30 minutes to 5 h, more particularly for 1 h to 3 h, in particular for 2 h, at a concentration of between 100 and 600 μM, in particular between 200 and 400 μM, in particular of 300 μM.

Step c) is carried out to determine cell viability. The techniques for determining cell viability are well known to those skilled in the art. Mention may in particular be made of the methylene blue staining technique, propidium iodide staining, or alternatively the lactate dehydrogenase (LDH) enzyme release test.

The method according to the invention can advantageously be used to carry out a high-throughput screening of several test compounds. Thus, the cells may in particular be cultured in devices suitable for such a high-throughput screening, in particular in multiwell plates, for example plates with at least 6 wells, at least 12 wells, at least 24 wells or at least 96 wells. According to a particular embodiment, the screening method according to the invention is carried out on a 96-well plate.

The test compounds selected according to the screening method described above can then be tested for their ability to induce autophagy and/or metabolic reprogramming. The evaluation of the capacity of the test compounds to induce autophagy can in particular be carried out according to the method described in the examples. In short, the steps of the screening method described above are carried out on cardiac cells in which the expression or activity of proteins involved in the autophagy mechanism is inhibited. If the selected compounds are no longer capable of inhibiting cell death under these conditions, it can be concluded that their cardioprotective activity depends on induction of autophagy. The inhibition of the expression or of the activity of proteins involved in the autophagy mechanism can be carried out by any means known in the art, in particular by transfection of an inhibitory nucleic acid, in particular a siRNA, targeting the mRNA encoding one or more proteins involved in the autophagy mechanism. Mention may be made in particular of the inhibition of the Atg5 protein or of the Beclin-1 protein, in particular by inhibition of their expression. According to one embodiment, the method comprises inhibiting Atg5 and Beclin-1. According to a particular embodiment, the method comprises inhibiting the expression of Atg5 and Beclin-1. In a more particular embodiment, the method comprises inhibiting Atg5 and Beclin-1 by transfection of siRNA.

The ability of the compounds to induce autophagy can also be evaluated by measuring the conversion of LC3 I and II in cells treated with the test compound selected by means of the screening method according to the invention. An increase in the conversion of LC3 I to LC3 II shows an induction of autophagy. Furthermore, the ability of the selected compounds to induce autophagosome formation may be determined in order to evaluate the ability of said compounds to induce autophagy.

The ability of the selected compounds to reprogram the energy metabolism can be determined by techniques known to those skilled in the art. It may thus be envisaged in particular to evaluate the capacity of the selected compounds at a high local level of reactive oxygen species in cardiac cells treated with the selected compound following the screening according to the invention. Those skilled in the art may advantageously use techniques for detecting the level of superoxide anion, in particular those based on the use of the MitoSOX fluorescent probe in confocal microscopy. In addition, the energy metabolism can also be analyzed in real time, in particular by measuring oxygen consumption proportional to mitochondrial respiration and measuring proton production and secretion in the culture medium proportional to glycolysis. Those skilled in the art have available to them methods allowing such an analysis, in particular by means of the Seahorse technology employed in the part used below and the measurement of the two ATP synthesis pathways mentioned above and called: oxygen consumption rate (OCR) and extracellular acidification rate (ECAR). These two parameters can be determined dynamically in the cells after sequential treatment with reference inhibitors of mitochondrial respiratory chain complexes (e.g. rotenone, antimycin, oligomycin).

The absence of toxic effects of the selected compounds can also be evaluated. Thus, the absence of proliferative effects can be evaluated according to techniques well known to those skilled in the art.

The cardioprotective capacities of the compounds selected by means of the screening method according to the invention can also be verified in animals.

Moreover, the effects of the test compounds can be compared to compounds of which the cardioprotective effects and properties of induction of autophagy and metabolic reprogramming are already known. In this respect, those skilled in the art may advantageously use one or more compounds chosen from SG6163F, VP331 and LOPA87, and analogs thereof, as reference in the screening method of the invention.

### FIGURE LEGENDS

FIG. 1. High-throughput screening of cardiac cell death inhibitors for hit identification.

FIG. 1A represents a flow chart of the screening.

FIG. 1B shows the ranking of the hits on the percentage survival revealed by methylene blue staining. The molecules were used at 10 μM in pretreatment before induction of cell death.

FIG. 1C represents the confirmation of the 6 best hits on the viability of H9C2 cells, measured by an LDH release assay.

Each experiment was reproduced at least 3 times. LB, Lysis Buffer. SD, Standard Deviation. ****, p<0.0001 relative to H₂O₂, ns., not significant. SD is calculated by single-factor ANOVA, with multiple Sidak comparisons.

FIG. 2. Cellular effects of the hits on primary rat neonatal ventricular cardiomyocytes (RNVCs) and lung cancer cell line (A549).

FIG. 2A shows that the hits protect the viability of RNVCs against H₂O₂ at 300 μM.

FIG. 2B shows that the hits protect the viability of RNVCs against camptothecin at 10 μM.

FIG. 2C shows the influence of hits on the viability of H₉C₂ cells. The cells were cultured in the presence of the hits at 10 μM for 48 h. Finally, the cells were lysed in lysis buffer and the amount of LDH was measured to evaluate the total cell growth.

FIG. 2D shows the influence of the hits on the viability of A549 cancer cells. The cells were cultured for 6 h in the presence or absence of the hits, and then for 42 h. Finally, the cells were lysed with lysis buffer and the amount of LDH was measured to evaluate the total cell growth.

Each experiment was reproduced at least 3 times. LB, Lysis Buffer. LDH, lactate dehydrogenase. The data are presented as mean±standard error of the mean (s.e.m) by single-factor ANOVA, with multiple Sidak comparisons. *, p<0.05, **, p<0.01, ***, p<0.001, ****, p<0.0001, with respect to 300 μM H₂O₂ (A), 10 μM camptothecin (B) or 0.01% DMSO (C).

FIG. 3. Effects on the expression of pro- and anti-apoptotic members of the Bcl-2 family

Levels of protein expression of BCL-2 (FIG. 3A and FIG. 3B) BXL-XL (FIG. 3C) and BAX (FIG. 3C), in the RNVCs after 6 h of treatment (FIG. 3A, FIG. 3C, FIG. 3D) or 24 h (FIG. 3B). Co., untreated cell control. Each experiment was reproduced at least three times. Representative western blotting images and quantification of three independent experiments are presented as mean±SEM with single-factor ANOVA, with multiple Sidak comparisons. *, p<0.05 with respect to DMSO.

FIG. 4. Inhibition of RNVC cell death by the hits requires Atg5 and Beclin-1

FIG. 4A: the RNVCs were transfected with siRNAs targeting Atg5 and Beclin-1, and the expression levels of the two proteins were evaluated by western blotting.

FIG. 4B and FIG. 4C: after transfection of Atg5 and Beclin-1 siRNA respectively, the LDH release was measured in the RNVCs treated with 300 μM H₂O₂, 2 h. Data are presented as mean +SEM with single-factor ANOVA, and multiple Sidak comparisons. ns., not significant with respect to cells treated with H₂O₂ and transfected with the siRNAs.

FIG. 4D: after transfection of the plasmid GFP-LC3, the fluorescent green GFP-LC3 spots were visualized by fluorescence microscopy and quantified with ImageJ to verify autophagosome formation. Data are presented as mean±SEM with single-factor ANOVA, and multiple Sidak comparisons. ***, p<0.001 with respect to 0.01% DMSO.

FIG. 4E: LC3-I/II protein levels in the RNVCs. Data are presented as mean±SEM with single-factor ANOVA, and multiple Sidak comparisons. *, p<0.05, **, p<0.01 with respect to DMSO.

FIG. 5. SG6163F stimulates autophagic flux in RNVCs

FIG. 5A: LC3-I/II and p62 protein levels in the RNVCs treated with 3-methyladenine (3MA), chloroquine (CQ) and SG6163F for 6 h. Rapamycin at 3 μM was used as positive control. Data are presented as mean±SEM with single-factor ANOVA, and multiple Sidak comparisons. *, p<0.05, **, p<0.01, ns., not significant.

FIG. 5B: after treatment with SG6163F, rapamycin, 3-methyladenine (MA), chloroquine (CQ), the fluorescent green GFP-LC3 spots were visualized by fluorescence microscopy and quantified with ImageJ. The data are presented in the form of mean +SEM with single-factor ANOVA, and multiple Sidak comparisons, *, p<0.05, **, p<0.01. The experiments were reproduced three times.

FIG. 6. Effects on mitochondrial dynamics by fluorescence microscopy The RNVCs were treated with 1 μM hits (FIG. 6A) and 10 μM hits (FIG. 6B) for 6 h and the mitochondrial network was stained with 200 nM of Mitotracker. The mitochondrial network and individual mitochondria were then analyzed using a Leica confocal microscope and IMARIS software. Data are presented as mean±SEM with single-factor ANOVA, and multiple Sidak comparisons. *, p<0.05, **, p<0.01, ***, p<0.001 with respect to DMSO.

FIG. 6C: MFN1 and MFN2 protein levels in RNVCs analyzed by western blotting.

FIG. 6D: Drp-1 and P-Drp-1 and MFN2 protein levels in RNVCs analyzed by western blotting. *, p<0.05, **, p<0.01, ***, p<0.001 with respect to DMSO. The experiments were reproduced 3 times. Representative western blotting images and quantification of three independent experiments are presented as mean±SEM with single-factor ANOVA, and multiple Sidak comparisons.

FIG. 7. Effects of SG6163F and digoxin on mitochondrial morphology and abundance by transmission electron microscopy.

The cells were treated with a vehicle (0.01% DMSO), 1 μM digoxin, 1 μM SG6163F, 10 μM SG6163F, fixed with glutaraldehyde and analyzed by transmission electron microscopy. The cells treated with digoxin and with 10 μM SG6163F have many shorter round mitochondria compared with the control which has long thin mitochondria (FIG. 7B, FIG. 7D versus FIG. 7A). Treatment with 1 μM SG6163F did not affect the morphology of the mitochondria (FIG. 7D versus FIG. 7C). The right insert in FIG. 7D shows a figure of mitochondrial fission.

FIG. 8. Metabolic reprogramming effects of the hits.

FIG. 8A: The H9c2 cells were treated with the compounds for 6 h, then the glycolytic function was measured using a glycolytic stress test on the XFe96 extracellular flow analyzer (Agilent, USA); the data are presented as mean±SEM with one-way ANOVA, and multiple Sidak tester comparisons. *, p<0.05, **, p<0.01, ***, p<0.001 with respect to DMSO.

FIG. 8B: the H9c2 cells were treated with the compounds for 6 h, then a mitochondrial respiration stress test was carried out.

FIG. 8C: the oxidation of the exogenous fatty acids was measured simultaneously using the XF palmitate-BSA FAO substrate kit with the mito stress test on XF cells. Before starting the test, 30 μL of XF palmitate-BSA CAM or BSA control substrate were added to the appropriate wells.

FIG. 8D: AMPK alpha2 and phospho-AMPK protein levels in RNVCs. Data are presented as mean±SEM with single-factor ANOVA, and multiple Sidak comparisons. *, p<0.05, **, p<0.01, ***, p<0.001 with respect to DMSO.

FIG. 9. Cellular effects of the compounds on RNVCs and H9c2.

The permeabilization of the RNVC cell membrane (FIG. 9A and FIG. 9B) was measured with propidium iodide. Data are presented as mean±SEM with single-factor ANOVA, and multiple Sidak comparisons. *, p<0.05, **, p<0.01, ***, p<0.001 with respect to DMSO.

FIG. 10. Cellular effects of analog compounds on RNVCs.

The protection of RNVC cells against H₂O₂ by SG6163F and LOPA87 analog compounds was measured using an LDH release assay and measurement of absorbance at 490 nm. Data are presented as mean±SEM with single-factor ANOVA, and multiple Sidak comparisons. *** , p<0.001 with respect to DMSO.

FIG. 11. Effects of the compounds on total cell volume.

FIG. 11: following the treatment of the RNVCs with the compounds as indicated, the cell volume was stained with 4 μM of calcein-AM. The cell volume was then analyzed using the IMARIS software.

FIG. 12. Effects of the compounds on mitochondrial production of ROSs.

FIG. 12: The RNVCs were treated with 0.1% DMSO, 3 μM rapamycin, 1 μM digitoxigenin, 1 μM digoxin, 1 μM minaprin, 1 μM VP331, 1 μM LOPA87, 10 μM SG6163F for 6 h, then the ROS production was stained with the MitoSOX fluorescent probe.

FIG. 12A: the fluorescence was captured with a Leica confocal microscope.

FIG. 12B: quantification of mitochondrial fluorescence intensity was performed with ImageJ software. The data are presented as mean±SEM with single-factor ANOVA, and multiple Sidak comparisons. *, p<0.05, **, p<0.01, ***, p<0.001 with respect to DMSO.

### EXAMPLES

### Materials and Methods

### High-Throughput Screening

Chemical libraries. The compounds were obtained from two chemical libraries: the Prestwick library (1200 molecules) and the CEA Saclay library (400 molecules). All compounds were supplied at 10 mM in 100% DMSO.

Cell treatment and induction of cell death. H9C2 cells were cultured in DMEM medium (ATCC® 30-2002™) supplemented with 10% fetal calf serum (BSA; BioWhittaker DE14-801F) and penicillin-streptomycin (Gibco® #15070063). The H9C2 cells were seeded in 96-well plates (5000 cells/well), allowed to adhere for 48 hours (h) and treated with compounds of the libraries at 10 μM for 2 h at 37° C. Next, the compounds were removed and replaced with a cell death inducer: treatment for 24 h with 10 μM of camptothecin (Sigma C9911) for apoptosis or treatment for 2 h with 300 μM of H₂0₂ (Sigma 216763) for necrosis.

Viability measurement and selection of hits. After treatment, the cells were washed twice with PBS and fixed with 95% ethanol for 30 minutes (min) at ambient temperature. The plates were emptied and dried overnight. The cells were then stained with methylene blue (0.1 g/l) for 5 minutes. After three washes with water, 0.1 M HCl was added to the plates. The absorbance reading was carried out at 665 nm (Envision spectrofluorimeter, Perkin Elmer). The results were standardized with a nontreated control and the hits were selected if the absorbance value was greater than the mean cell death control value+3 standard deviations.

### Isolation of Neonatal Cardiomyocytes

Neonatal rat cardiomyocytes were isolated as described previously (Wang et al., Cell Death Dis, 2016, 7:e2198).

### LDH Release Assay

A colorimetric assay was used to measure lactate dehydrogenase (LDH), a cytosolic enzyme which is released during permeabilization of the plasma membrane (Promega G1780). The assay was performed using cell culture supernatants obtained from H9C2 or RNVC (rat neonatal ventricular myocyte) cells and lysis buffer (LB) was used as a positive control for total cell lysis. The LDH release was measured by absorbance reading at 490 nm (Infinite spectrofluorimeter, Tecan).

### Cell Plasma Membrane Permeabilization Test

Propidium iodide, a non-permeable fluorescent DNA marker, was used to measure membrane integrity. Propidium iodide at 10 μM was added to the culture medium and a fluorescence reading was performed (for example: 530 nm; em: 620 nm), the LB was used as a positive control for total cell lysis.

### Pharmacology

The evaluation of the binding of the compounds to the human hERG potassium channel was carried out as described previously (Eurofins).

### Transfection of Plasmids

On day 0, 4×10⁵ neonatal cardiomyocytes were plated overnight in laminin-coated 35 mm culture dishes (10 μg/ml). On day 1, the cells were transfected with 1 μg of GFP-LC3 plasmid using the transfection reagent Lipofectamine® 2000 (1 μg of GFP-LC3 corresponds to 2.5 μl of transfection reagent), for 48 h, then the green fluorescence was detected with a Leica confocal microscope (SP5). The analyses were carried out with the ImageJ program (Wayne Rasband, National Institutes of Health, USA).

### Analysis of the Mitochondrial Network by Microscopy

Confocal microscopy. On day 0, 4×10⁵ neonatal cardiomyocytes were plated overnight on laminin-coated 35 mm culture dishes (10 μg/ml). On day 1, the cells were treated with various compounds at 1 or 10 μM for 6 hours. The cells were incubated with Mitotracker Red 580 at 200 nM for 20 minutes at 37° C., then 4 μM of calcein (Calcein AM, Life Technologies, St Aubin, France) for 10 minutes at 37° C. The images were acquired with a Leica confocal microscope (SP5) (Mannheim, Germany). The mitochondrial network and the 3D model of cell volume were reconstructed using the IMARIS software (Bitplane Company, Zurich, Switzerland); therefore the cell volume, mitochondrial number and volume were analyzed.

Transmission electron microscopy. For ultrastructural analysis, the cells were fixed in 1.6% glutaraldehyde in a 0.1 M phosphate buffer, rinsed in a 0.1 M cacodylate buffer, and post-fixed for 1 h in 1% osmium tetroxide and 1% potassium ferrocyanide in a 0.1 M cacodylate buffer to improve the coloration of the membranes. The cells were rinsed in distilled water, dehydrated in alcohols and finally embedded in epoxy resin. Contrasting ultrafine sections (70 nm) were analyzed under a JEOL 1400 transmission electron microscope with a Morada Olympus CCD camera.

### Detection of ROSs in RNVCs

The RNVCs were treated with 0.01% DMSO, 3 μM rapamycin, 1 M digitoxigenin, 1 μM digoxin, 1 μM minaprine, 1 μM VP331, 1 μM LOPA87, 10 μM SG6163F in a serum-free cell culture medium for 6 hours. The contents (50 μg) of a MitoSOX™ mitochondrial superoxide indicator vial (ThermoFisher, M36008) were dissolved in 13 μl dimethyl sulfoxide (DMSO) to prepare a 5 mM MitoSOX™ reagent stock solution. Next, the 5 mM MitoSOX™ reagent stock solution in PBS was diluted to prepare a 5 μM working solution of MitoSOX™ reagent. After treatment, the cells were rinsed twice with hot PBS and then incubated with a MitoSOX reagent working solution for 10 minutes at 37° C. The cells were gently rinsed three times with hot PBS. Red fluorescence was detected with a Leica confocal microscope. Nuclear fluorescence was suppressed and mitochondrial fluorescence intensity was measured using ImageJ software.

### Real-Time Bioenergetic Profile Analysis of H9C2 Cardiomyocytes

The XFe96 extracellular flow analyzer (Seahorse Biosciences, North Billerica, MA, USA) was used to measure the bioenergetic function of H9C2 cardiomyocytes. The H9C2 cells were seeded at 20 000 cells per well in XF96 cell culture microplates; all the pretreatments were carried out with serum-free cell culture medium. The Agilent Seahorse XF glycolysis stress test was performed to measure glycolytic function in the H9C2 cells; the extracellular acidification rate (ECAR) was measured sequentially by 3 injections, 10 mM glucose, 2 μM oligomycin and 50 mM 2-deoxy-D-glucose (2-DG). The Agilent Seahorse XF Cell Mito stress test measured key parameters of mitochondrial function by directly measuring the oxygen consumption rate (OCR) of the H9C2 cells. The test used the built-in injection ports on XF sensor cartridges to add respiration modulators into the cell well during the test in order to reveal key parameters of mitochondrial function. Oligomycin at 2 μM was injected first after the baseline measurements. Then 1 μM of 4-carbonyl cyanide (trifluoromethoxy) phenylhydrazone (FCCP) was injected. The third injection corresponds to 0.5 μM of antimycin A, to stop mitochondrial respiration. The oxidation of the exogenous fatty acids was measured simultaneously using the XF palmitate-BSA FAO substrate kit with the mitochondrial stress test on XF cells. The substrate-limited medium is DMEM with 0.5 mM glucose, 1 mM GlutaMAX, 0.5 mM carnitine and 1% fetal calf serum. Carnitine was added freshly on the day of the change of medium. The FAO test medium contains 111 mM NaCl, 4.7 mM KCl, 1.25 mM CaCl₂, 2 mM MgSO₄, 1.2 mM NaH₂PO₄, supplemented with 2.5 mM glucose, 0.5 mM carnitine and 5 mM HEPES on the day of the test, adjusted to pH 7.4 at 37° C. The H9C2 cells were seeded at 20 000 cells per well in XF96 cell culture microplates. All pretreatments were carried out with serum-free cell culture medium. 24 hours before the assay, the growth medium was replaced with a substrate-limited medium. 45 minutes before the assay, the cells were washed twice with FAO test medium, 150 μl/well of FAO assay medium were added to the cells. The incubation was carried out in a CO₂-free incubator for 30 to 45 minutes at 37° C. The assay cartridge was loaded with XF Cell Mito stress test compounds (final concentrations: 2 μM oligomycin, 1 μM FCCP, 0.5 μM antimycin A). Just before starting the test, 30 μl of XF palmitate-BSA FAO or BSA substrate were added to the appropriate wells, then the XF cell culture microplate was immediately inserted into the XFe96 analyzer for analysis.

### SDS-PAGE and Western Blotting

The H9C2 cells and RNVCs were detached in LB containing: 50 mM Tris, 150 mM NaCl, 1 mM EDTA, 0.5% deoxycholate, 1% Triton X 100, 0.1% SDS (pH 8.0). The cells were collected and placed on ice for 30 minutes. The cells were then centrifuged at 2000 g for 20 minutes at for 4° C. The supernatant was transferred to a new tube and stored on ice. The protein concentration was determined by BCA assay. The protein samples were diluted with sample buffer (2× Laemmli, Sigma), mixed, and heated for 5 minutes at 95° C. The samples were then loaded into a precast gel with a gradient of 4 to 20% and migrated for 15 minutes at 300 V. After migration, the membrane and the gel were placed on the base of a Trans Blot Turbo cassette (Bio Rad) and the proteins were transferred for 3 minutes at 2.5 V. After transfer, the membrane was blocked with 5% milk in PBS-Tween and incubated with a primary antibody diluted in PBS-Tween containing 5% w/v milk at 4° C. with gentle stirring, overnight. The following day, the membrane was washed with PBS-Tween for 6×5 min and incubated with a secondary antibody conjugated to horseradish peroxidase for 1 h at ambient temperature. The membrane was washed again with PBS-Tween for 6×5 minutes after the secondary antibody. The membrane was then incubated with an enhanced ultra-sensitive chemiluminescent substrate for 5 minutes. The images were recorded with a gel imaging system (Bio Rad). For protein detection, the following antibodies were used: Anti-Mitofusin 1 (ab126575, Abcam, USA), anti-Mitofusin 2 (ab124773, Abcam, USA), BCL-2 (C-2) (sc-7382, Santa Cruz, USA), BAX (B-9) (sc-7480, Santa Cruz, USA), BCL-XL (#2764, Cell Signaling, USA), AMPK alpha2 (#2757, Cell Signaling, USA), LC3B (D11) (#3868, Cell Signaling, USA), ß-Actin (C4) (sc-47778, Santa Cruz, USA), Phospho-DRP1 (Ser616) (D9A1) (#4494, Cell Signaling, USA), DLP1 (#611112, BD Biosciences, USA).

### Quantitative RT-PCR

The total RNA was purified from 0.5 million rat H9C2 cells using the RNeasy Mini Kit (Qiagen). The RNA amounts were quantified using the NanoDrop 2000 spectrophotometer (Thermo Scientific). The RNA integrity was verified using the Agilent 2100 Bioanalyzer with the RNA 6000 Nano test (Agilent Technologies). 1 μg of total RNA was reverse-transcribed in a final reaction volume of 20 μl using the High Capacity cDNA Reverse Transcription kit (Life Technologies) with an RNase inhibitor and random primers according to the manufacturer's instructions. The quantitative PCR was performed with the QuantStudio 12K Flex real-time PCR system using the TaqMan detection protocol. 6 ng of cDNA were mixed with TaqMan Universal Master Mix 2 and each TaqMan® test in a final volume of 10 μl. The reaction mixture was loaded onto 384-well microplates and subjected to 40 PCR cycles (50° C./2 min; 95° C./10 min; (95° C./15 s; 60° C./1 min)×40). A qPCR analysis in the absence of a reverse transcription step was performed on all the RNA samples to verify the absence of any DNA contamination. Each sample measurement was made in duplicate and three independent RNA biological samples were prepared for each condition. The Ct values were then used for the quantification, and the relative gene expression ratio was determined using the ΔΔCt method and standardized by means of the geometric mean of a set of stable domestic genes.

### Statistical Analyses

The results are expressed as the mean +standard error of the mean (s.e.m). The Origin and Graphpad Prism 6 software were used for the statistical analysis. Differences between 2 groups were analyzed by single-factor ANOVA and differences between the groups of two genotypes were analyzed by two-way ANOVA, with multiple Sidak comparisons. The statistical significance is indicated as follows: * P<0.05, ** P<0.01, *** P<0.001, **** P<0.0001. The number of cells and of independent experiments performed is indicated in the figure legends.

### Results

### 1. Identification of Cardiomyocyte Death Inhibitors by High-Throughput Screening

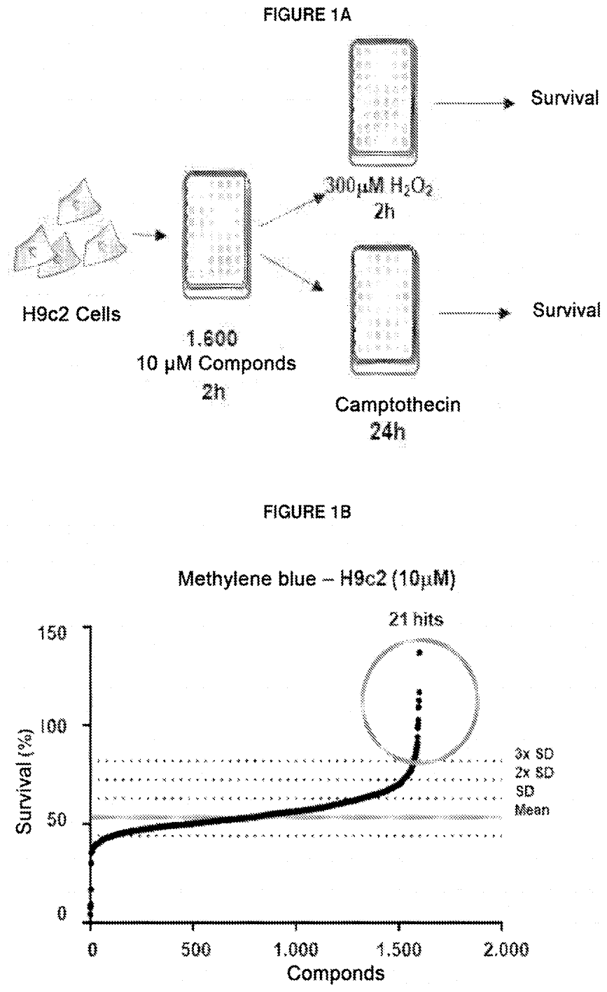

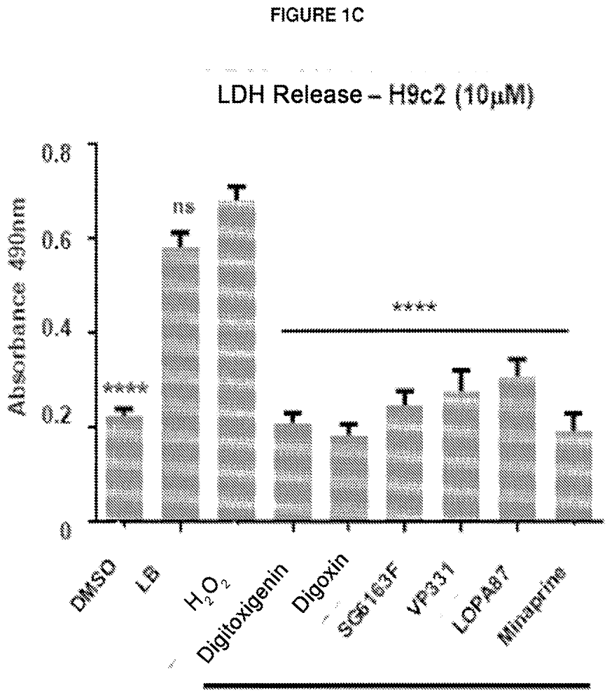

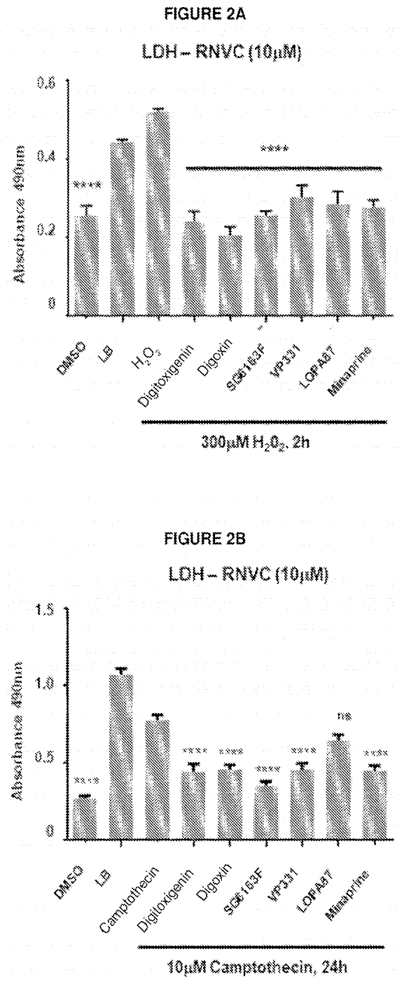

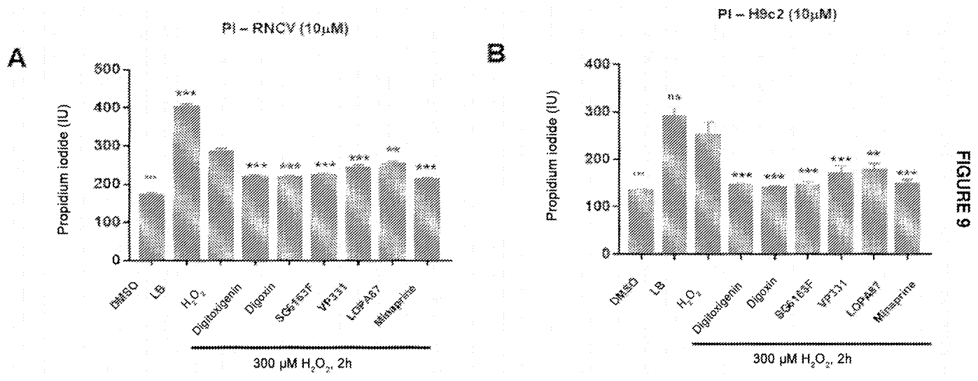

Two high-throughput screens were developed to identify inhibitors of cell death due to H₂O₂-induced necrosis and to camptothecin-induced apoptosis, in the Prestwick commercial library (1200 molecules) and a CEA Saclay house library (400 molecules). The H9C2 cardiomyoblasts were pretreated with the compounds at 10 μM for 2 h with 0.01% DMSO as vehicle, washed and incubated with a cell death inducer (i.e. H₂O₂ or camptothecin) for the period indicated (FIG. 1A). Then, the percentage of viable cells was evaluated by methylene blue staining, and the hits were classified according to their effectiveness in protecting against cell death, revealing 21 statistically significant hits (FIG. 1B). Next, 6 hits were confirmed manually on H9C2 cells with new batches of molecules using the lactate dehydrogenase (LDH) release test (FIGS. 1C, 1D) and propidium iodide staining (FIG. 9) independently. Three hits belong to the Prestwick library, namely digitoxigenin, digoxin and minaprine, and 3 hits belong to the CEA bank: SG6163F, VP331 and LOPA87 (Gabillet, Loreau et al. 2014). The chemical structures of the 6 hits are shown in FIG. 1E. Some analogs of SG6163F and LOPA87 were then analyzed for their ability to protect against the effects of H₂O₂ and showed efficacy.

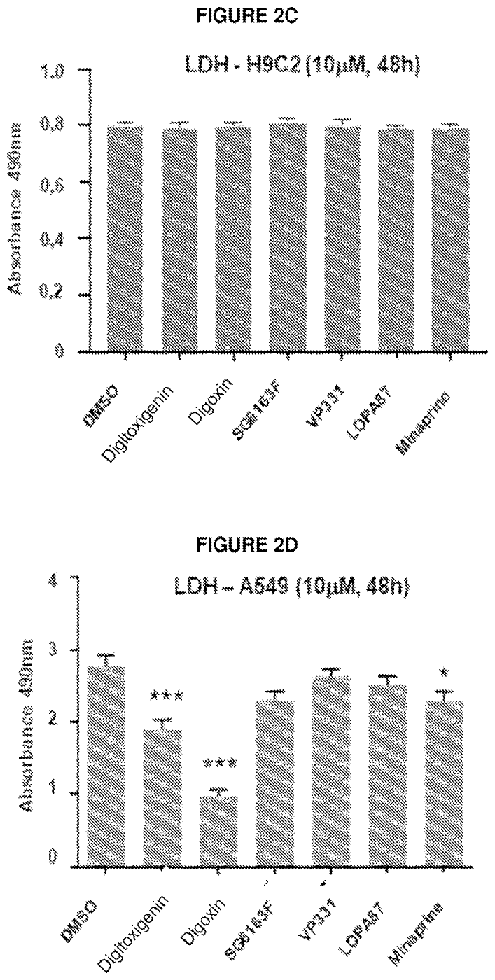

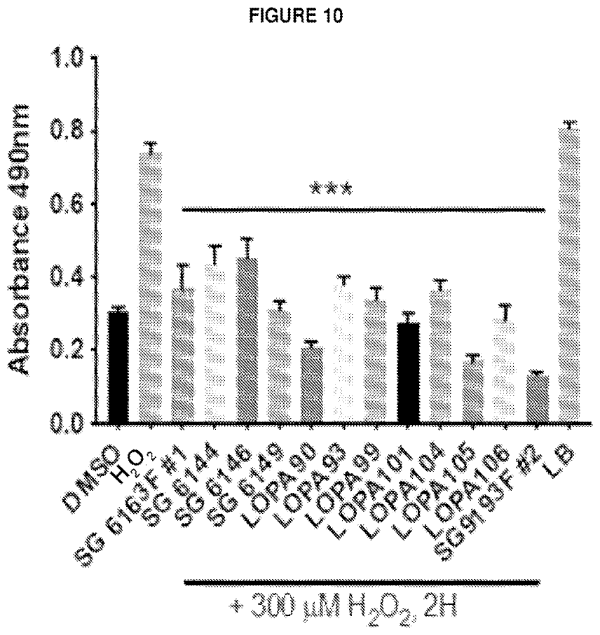

The effect of the compounds was then evaluated on primary rat neonatal ventricular cardiomyocytes (RNVCs) using the LDH test and propidium iodide staining. The 6 compounds were found to effectively inhibit both necrosis and apoptosis of RNVCs, with the exception of LOPA87, which did not protect against camptothecin-induced apoptosis when detected by LDH release (FIGS. 2A, 2B, FIG. 10), without detectable toxicity at 48 h in H9C2 cells (FIG. 2C). The absence of proliferative effects of the compounds was also evaluated on the A549 lung cancer cell line for 48 h (FIG. 2C). Thus, digitoxigenin, digoxin and minaprine significantly reduced cell growth and induced cell death, whereas SG6163F, VP331 and LOPA87 showed no proliferative effect on A549 cell growth during this period (FIG. 2C).

Since the blocking of the human potassium channel hERG channel may be responsible for cardiac fibrillation leading to cardiac arrest and death of the patient, the binding of the 3 molecules SG6163F, VP331 and LOPA87 to this receptor was evaluated. To do this, the ability of the molecules to inhibit the specific binding of [3H]dofetilide to the channel in vitro was measured and it was found that the binding of SG6163F, VP331 and LOPA87 is negligible (table 1).

TABLE 1
hERG channel binding assay Inhibition of titriated
dofetilide binding was measured in duplicate in two
independent measurements and the mean was calculated.
Only values >50% are considered effective

| | Concentration | % inhibit | | |
|---|---|---|---|---|
| Compound | (μM) | 1st assay | 2nd assay | Mean |
| SG6163F | 10 | 40.5 | 36.6 | 38.5 |
| VP331 | 10 | −4 | 5.4 | 0.7 |
| LOP87 | 10 | 14.6 | 5.3 | 10 |

### 2. Effects of the Compounds on the Expression of Members of the BCL-2 Family

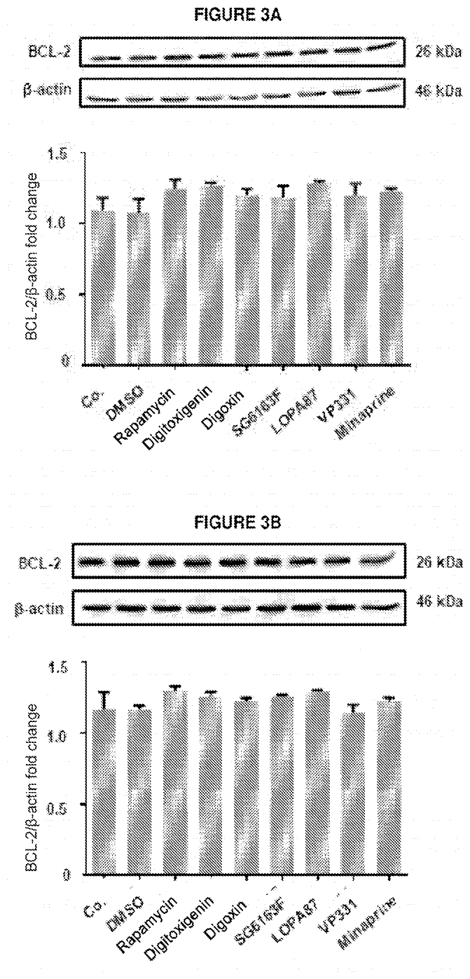

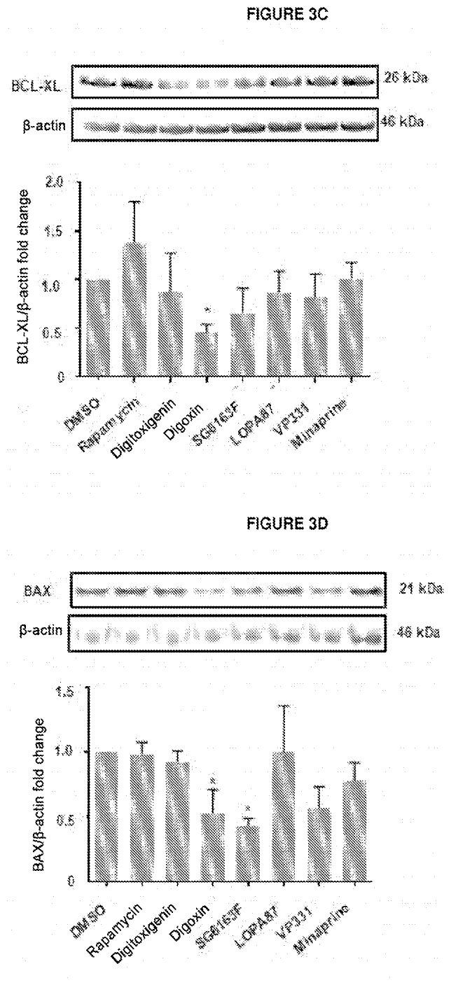

To determine the cellular mechanisms by which the identified compounds inhibit cell death, the expression level of members of the BCL-2 anti-apoptotic family after a short (6 h) or longer (24 h) treatment of RNVCs was determined. The compounds did not modify the expression of BCL-2 independently of the duration of the treatment (FIGS. 3A, 3B), whereas digoxin caused a decrease in the expression of BCL-XL (FIG. 3C). In addition, digoxin and SG6163F significantly decreased the expression of the pro-apoptotic BAX protein (FIG. 3D). To compare the results with reference molecules, use was made of rapamycin, a serine/threonine kinase inhibitor of mTOR and an autophagy inducer (Foster et al., 2010, Journal of Biological Chemistry 285(19): 14071-14077), which showed no effect on the expression of BCL 2 family members in RNVCs (FIG. 3).

### 3. Atg5- and Beclin-1-Dependency of Hits and Induction of Autophagy by SG6163F

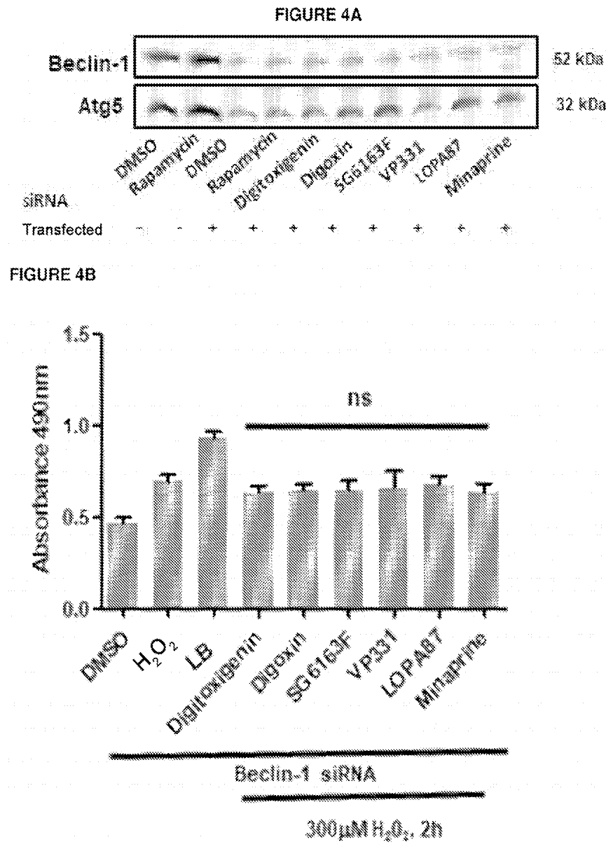

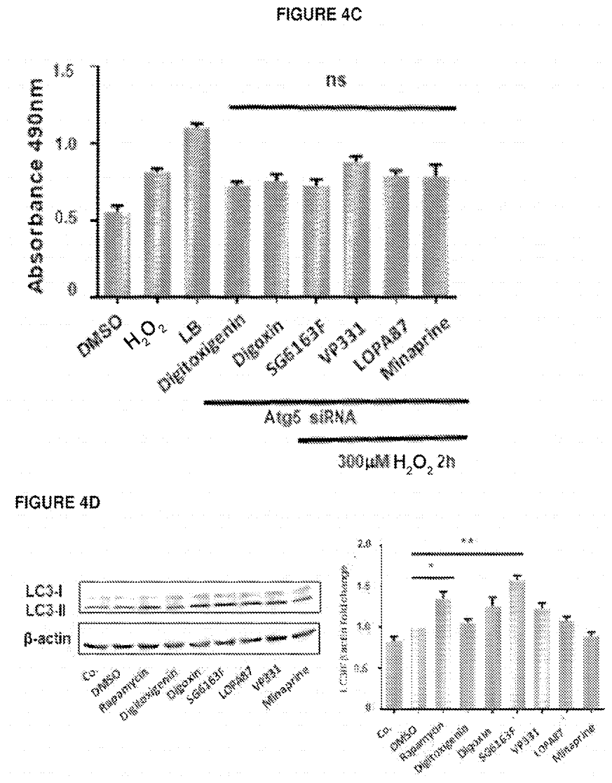

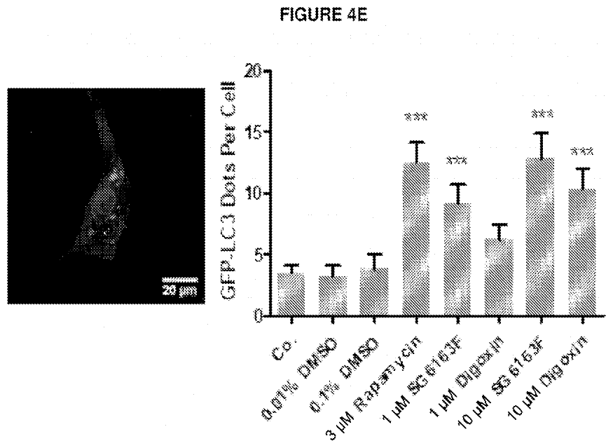

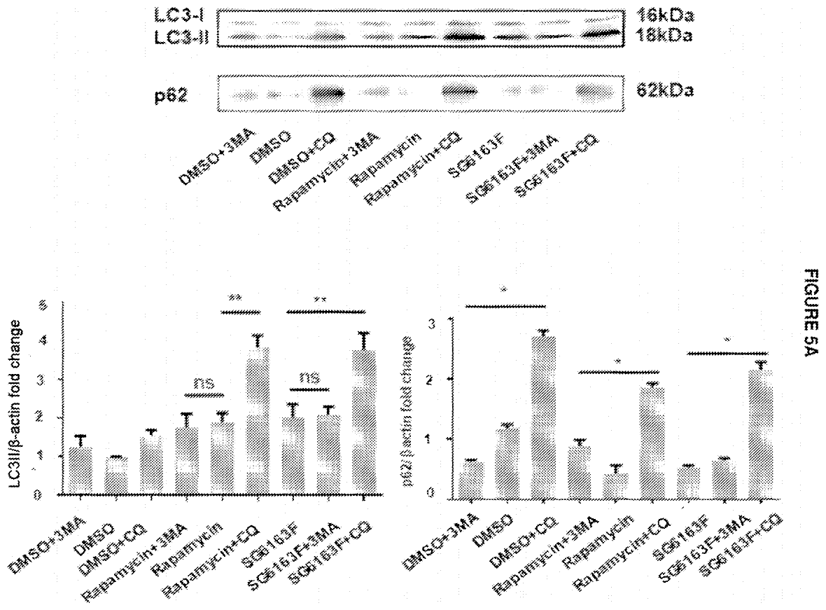

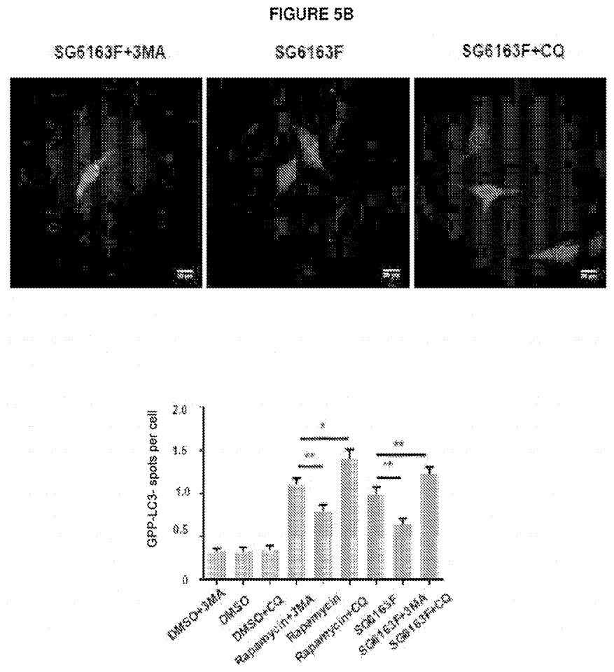

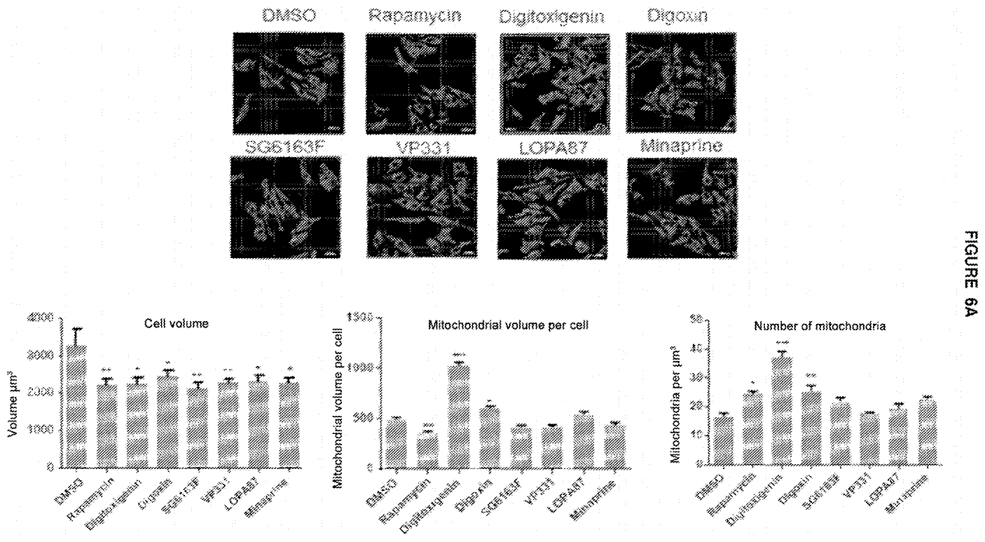

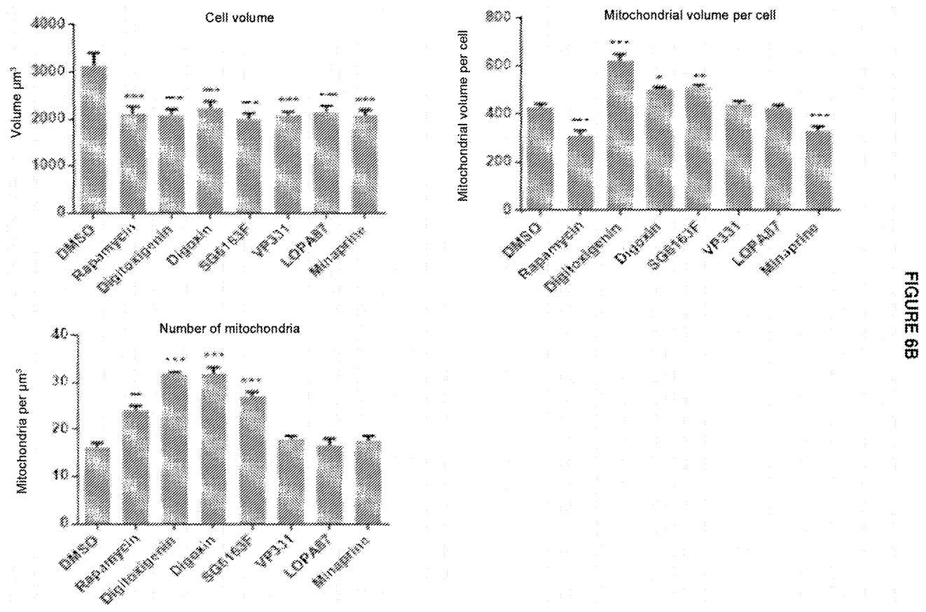

Next, the hypothesis that the compounds could protect cardiac cells by activating autophagy, a catabolic process conserved in evolution that involves the degradation of damaged cytoplasmic components, was formulated. Thus, to explore the dependency of hits on the machinery of autophagy proteins (Yin et al., 2016, Cell Death Dis.7:e2198), the expression of Atg5 and Beclin-1, two main participants in autophagy (Kang et al., 2011, Cell death and differentiation 18(4): 571), was inhibited by transfection of siRNA into RNVCs for 24 hours (FIG. 4A). Then, the cells were treated with the hits and with H₂O₂ for 2 hours and their survival was analyzed 4 hours later by measuring LDH release (FIGS. 4B, 4C). The results show that all compounds lost their ability to protect RNVCs from necrosis. All compounds also lost their ability to inhibit camptothecin-induced cell death (not shown). Since these results suggested that the hits had the ability to induce autophagy as cardioprotective activity, the conversion of LC3 I and II was measured and it was found that the level of expression of LC3II was significantly increased by treatment of cells with 1 μM SG6163F (>1.5-fold) and rapamycin (>1.4-fold), as shown by western blot analysis (FIG. 4D). In order to confirm the induction of autophagy by another approach, the RNVCs were transfected with a GFP-LC3 plasmid and the formation of the autophagosome was monitored 24 hours later. The results presented in FIG. 4E show that SG6163F is capable of causing the formation of autophagosomes. Rapamycin was used at 3 μM as a positive control for autophagy induction. Then, in order to specify the mechanisms of SG6163F activity, the autophagic flux was measured using two reference inhibitors, 3-methyladenine (3MA) and chloroquine (CQ), using the LC3 I/II western blotting technique (FIG. 5A) and the fluorescence microscopy technique after transfection of GFP-LC3 (FIG. 5B).

Overall, these results reveal that all the hits require Atg5 and Beclin1 to exert their cell death inhibitory activity, and stimulate autophagic flux through post-transcriptional mechanisms.

### 4. Impact on Mitochondrial Network and Reactive Oxygen Species in RNVCs

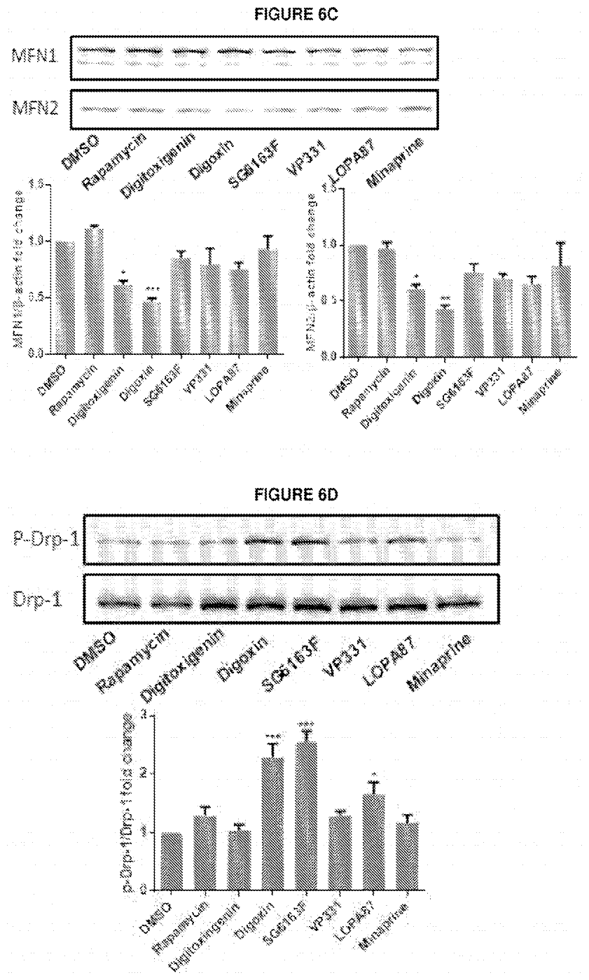

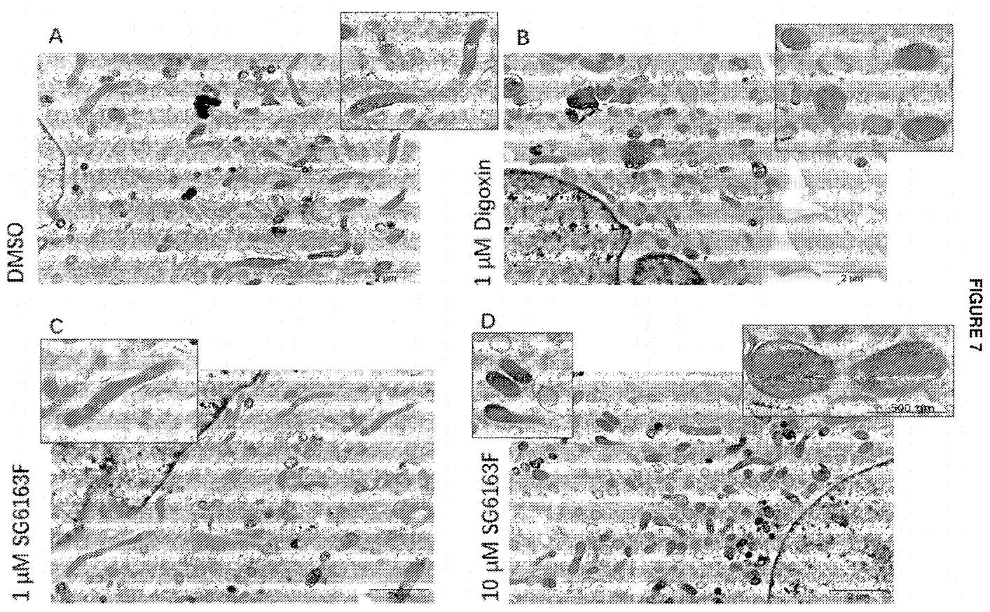

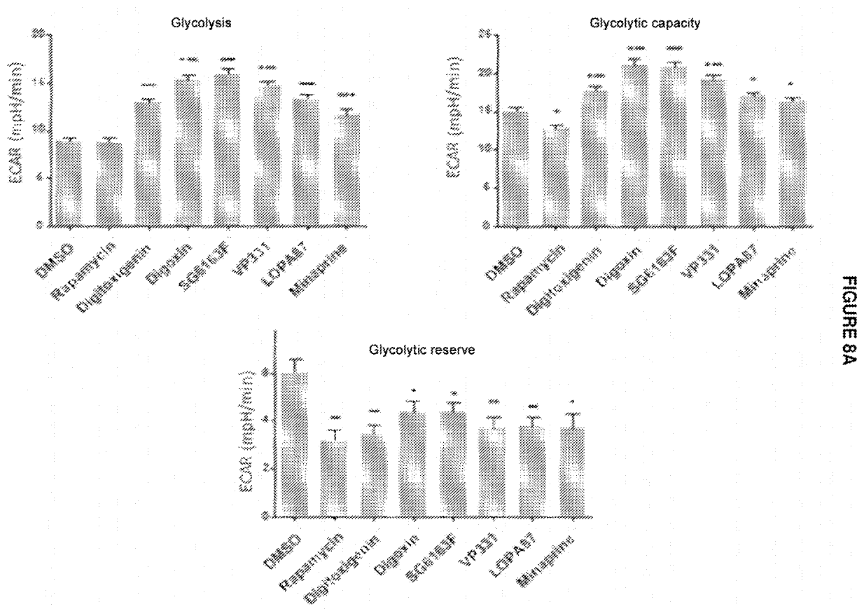

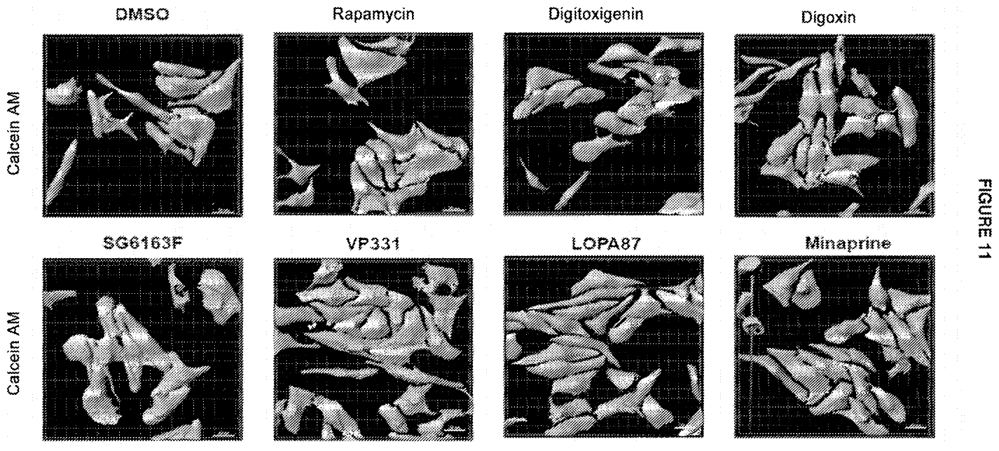

Due to the importance of mitochondria and reactive oxygen species (ROS) levels in the fate of cardiomyocyte cells, the hypothesis that the compounds could impact the mitochondrial network was explored. Following treatment of the RNVCs with the compounds tested, the mitochondrial network was stained with 200 nM of Mitotracker and the cell volume with 4 μM of calcein-AM. Thus, the mitochondrial network and the individual mitochondria were then analyzed using the IMARIS software. Although all the compounds significantly decreased the cell volume relative to the vehicle (FIG. 11), at 1 and 10 μM, digitoxigenin, digoxin and SG6163F increased the number of mitochondria and the total mitochondrial volume per cell, suggesting induction of mitochondrial biogenesis (FIGS. 6A, B). On the other hand, VP331, LOPA87 and minaprine had no effect on the mitochondrial network and the number of mitochondria, with the exception of 10 μM minaprine and 3 μM rapamycin, which decreased the mitochondrial mass (FIGS. 6A, B). Consequently, digitoxigenin and digoxin decreased the expression of the fusion proteins MFN1 and MNF2 (FIG. 6C), and digoxin and SG 6163F stimulated fission by phosphorylation of DRP-1 at the level of Ser616 as shown by the measurement of the (DRP1-P)/(DRP-1 total) ratio by western blotting (FIG. 6D). The effects on mitochondrial dynamics were confirmed by transmission electron microscopy showing an increase in the number of mitochondria and a decrease in their size in H9C2 cells treated with 10 μM SG6163F and 1 μm digoxin compared to mitochondria treated with DMSO and 1 μM of SG6163F (FIG. 7).

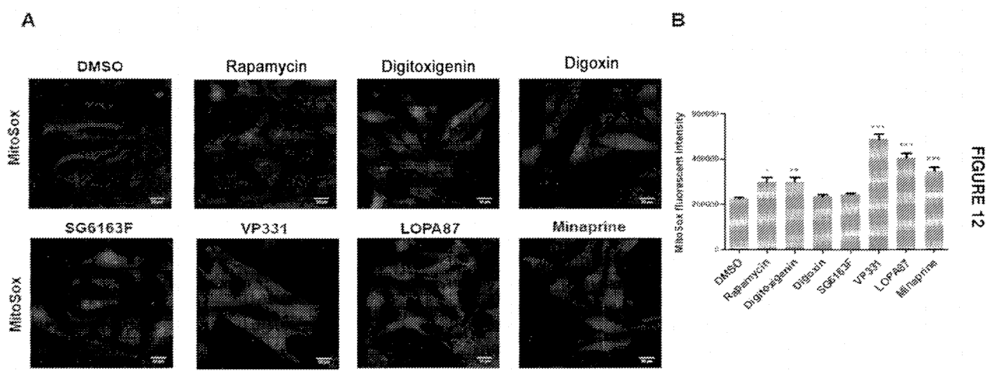

ROSs are produced or accumulated as a by-product of mitochondrial activity metabolism. Thus, the ROSs may be a consequence of mitochondrial function or may reflect a defect in the mitochondrial antioxidant system. Herein, using superoxide anion staining in RVNCs with the MitoSOX fluorescent probe after cell treatment with the compounds for 6 h, it was possible to show that rapamycin, digitoxigenin, VP331, LOPA87 and minaprine induced an elevation of the local level of mitochondrial superoxide anion, but digoxin and SG6163F did not (FIG. 12).

### 5. The Hits Identified Reprogram the Energy Metabolism of H9C2 Cells

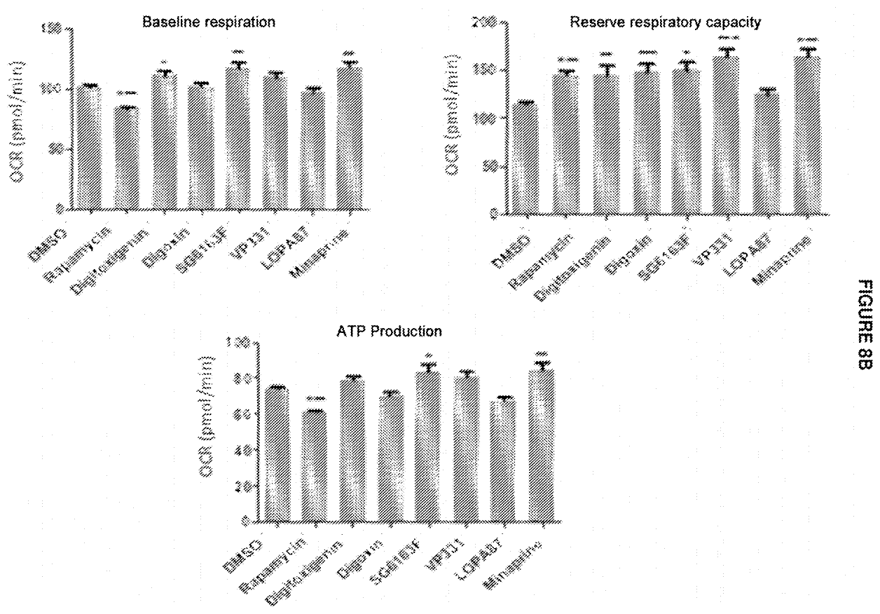

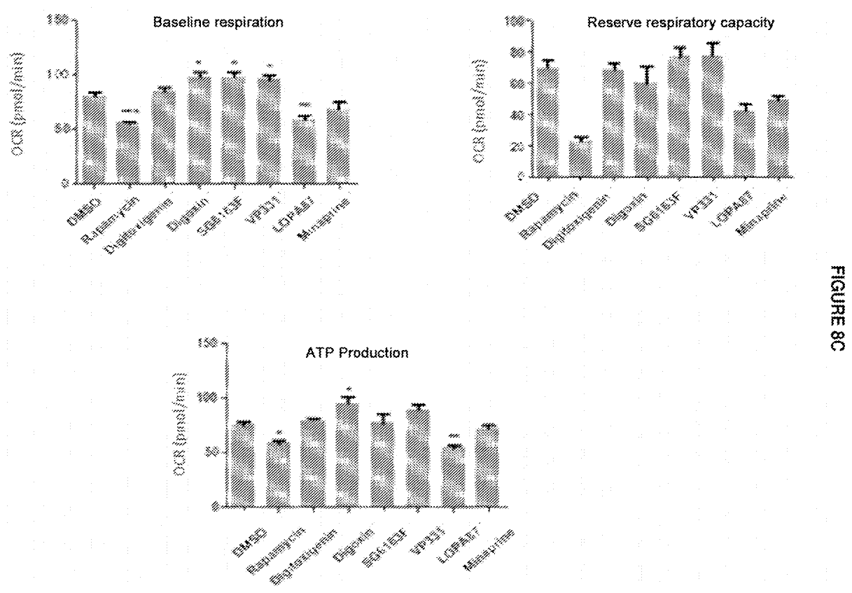

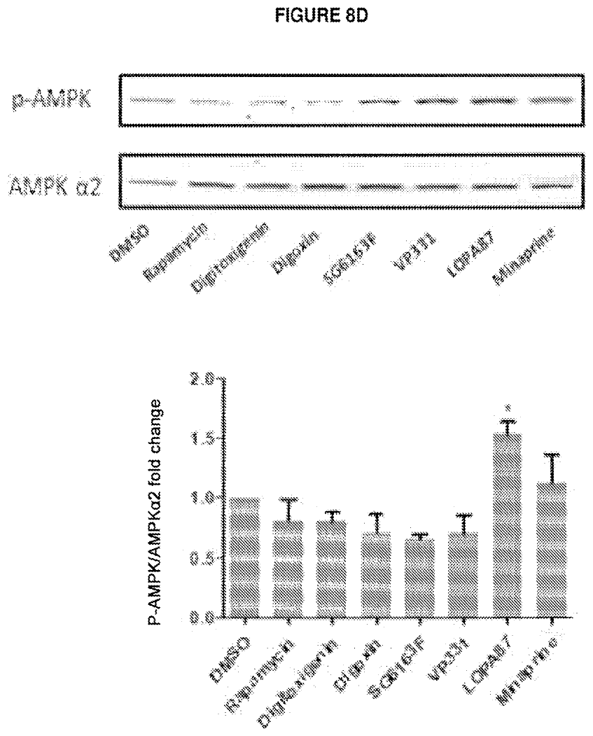

Next, the energy metabolism was analyzed in real time using the Seahorse technology in H9C2 cells. Firstly, all the compounds promoted extracellular acidification (increase in the ECAR) suggesting an increase in ATP production by anaerobic glycolysis, with the exception of rapamycin (FIG. 8A). Digitoxigenin and minaprine improved ATP production by oxidative phosphorylation (OXPHOS) using glucose and pyruvate as substrates (FIG. 8B), but not fatty acids (FIG. 8C). VP331 and digoxin improved oxidative phosphorylation using fatty acids as substrate (FIG. 8C), but not glucose (FIG. 8B). SG6163F was found to boost OXPHOS although rapamycin decreased it, independently of the substrates as expected (Schieke et al., 2006, Journal of Biological Chemistry 281(37): 27643-27652) (FIGS. 8B, C). Finally, in order to specify the metabolic reprogramming mechanisms, the activation of AMP-activated protein kinase (AMPK), a master regulator of metabolism, was studied and it was found that only LOPA87 stimulates AMPK phosphorylation 1.5-fold compared with the DMSO control (FIG. 8D).

### CONCLUSION

Two high-throughput screening assays for compounds that inhibit cell death were developed. These tests made it possible to identify 6 compounds that inhibit cardiac cell death by autophagy induction and metabolic reprogramming. Three molecules of interest (SG6163-F, LOPA87 and VP331) were more particularly identified and were validated manually in the H9C2 cell line and in rat neonatal ventricular myocytes (RNVCs) using lactate dehydrogenase release measurement and the fluorescent propidium iodide permeability assay. In addition, for each molecule, the involvement of pro-survival mechanisms was identified, such as Atg5 and Beclin-1 proteins of the autophagy machinery, modulation of BCL-2 oncoprotein expression, impact on mitochondrial network and mitochondrial biogenesis, production of reactive oxygen species and metabolic reprogramming. The new molecules were compared to four commercial molecules, 3 of which were identified in the screen, namely digitoxigenin, digoxin and minaprine, and a commercial molecule which is a control autophagy inducer, rapamycin. The 3 molecules of interest did not show cytotoxic activity on cardiomyocytes, did not increase the proliferation of cancer cells and did not bind to the hHERG cardiac canal. Their mechanisms of action are different than those of rapamycin. Thus, the molecules identified can be used in combination with chemotherapy or radiotherapy, in particular, to protect cardiac cells and to reduce the side effects of anticancer treatments.

1-11. (canceled)

12. A method of inducing a cardioprotective effect in a patient comprising administering a compound selected from the group consisting of SG6163F, LOPA87, VP331, and analogs thereof, to a patient in need of cardioprotection.

13. The method according to claim 12, wherein the compound is selected from SG6163F and LOPA87, and analogs thereof.

14. The method according to claim 13, wherein the compound is selected from SG6163F and LOPA87.

15. The method according to claim 12, wherein the patient in need of cardioprotection has a heart disease chosen from myocardial infarction, ischemia-reperfusion or heart failure.

16. The method according to claim 12, wherein the patient in need of cardioprotection is being treated with a therapy inducing cardiotoxic side effects.

17. The method according to claim 16, wherein the compound is administered before, during or after the therapy inducing cardiotoxic side effects.

18. The method according to claim 16, wherein the cardiotoxic side effect-inducing therapy is an anticancer therapy.

19. A kit of components comprising:
(a) a compound selected from the group consisting of SG6163F, LOPA87 and VP331, and analogs thereof; and
(b) a therapeutic agent useful in treating a patient, said agent however inducing cardiotoxic side effects;
wherein component (a) is used for its cardioprotective effect against the cardiotoxic side effects of component (b).

20. The kit of components according to claim 19, wherein components (a) and (b) are suitable for sequential, separate and/or simultaneous administration.

21. The kit of components according to claim 19 for use in the context of the treatment of cancer, component (b) being a chemotherapeutic or immunotherapeutic anticancer agent, and component (a) being used for its cardioprotective effect against the cardiotoxic side effects of component (b).

22. A method for screening for compounds which have cardioprotective activity, said method comprising the following steps:
(a) culturing cardiac cells in a culture medium containing a test compound;
(b) culturing the cardiac cells in a culture medium containing a cell death inducer; and
(c) measuring cell viability.
